(19) United States
(12) Patent Application Publication
(10) Pub. No.: US 2023/0020866 A1
(43) Pub. Date: Jan. 19, 2023

(54) METHOD AND SYSTEM FOR IDENTIFYING CANCER TWIN

(71) Applicant: Innoplexus AG, Eschborn (DE)

(72) Inventors: Dhiraj Jadhao, Pune (IN); Chiara Thanner, Frankfurt (DE)

(21) Appl. No.: 17/377,811

(22) Filed: Jul. 16, 2021

(51) Int. Cl. G16H 50/70; G16H 10/60

(52) U.S. Cl. CPC G16H 50/70; G16H 10/60

(57) ABSTRACT

There is disclosed a system for identifying at least one relevant subject for a cancer patient comprising a server arrangement communicably coupled to a database arrangement comprising a plurality of records and a user device of the cancer patient, wherein the server arrangement is configured to create a profile for the cancer patient by receiving inputs from the user device, map soft and hard attributes of the cancer patient with pre-existing profiles present in the database arrangement, list the at least one relevant subject for the cancer patient, and connect the cancer patient with the listed at least one subject for information sharing purposes.

The present disclosure relates generally to system for identifying patients of interest, and more specifically, to system and method for identifying at least one relevant subject for a cancer patient.

### BACKGROUND

Cancer, a leading fatal disease, features an abnormal mass of malignant tissue resulting from excessive cell division. Cancer cells proliferate in defiance of normal restraints on cell growth, and invade and colonize territories normally reserved for other cells. Conventional treatment protocols for cancer include chemotherapy, surgery, radiation, and combinations of these treatments.

Moreover, a patient going through any conventional or experimental treatment protocol of cancer has to face lots of mental trauma/stress. The biggest reason of facing mental trauma is unavailability of correct and relevant information to various patients.

Conventionally, the information available on the internet is scattered in bites and pieces, also, the accuracy of the available information is always questionable as it is not validated. Thus, a person facing issues related to cancer always face challenge in opting a treatment protocol for cancer.

Moreover, the experience of cancer survivors and patients going through any treatment protocol is very useful for new patients and helps patients to reduce mental trauma or stress.

Therefore, in light of the foregoing discussion, there exists a need to overcome the aforementioned drawbacks associated with existing information retrieval system for cancer patients.

### SUMMARY

The present disclosure seeks to provide a system for identifying at least one relevant subject for a cancer patient. The present disclosure also seeks to provide a method for identifying at least one relevant subject for a cancer patient. The present disclosure seeks to provide a solution to the existing problem of unmanageable, unstructured, time consuming and inefficient techniques of information retrieval system for cancer patients.

An aim of the present disclosure is to provide a solution that overcomes at least partially the problems encountered in prior art, and provides processing and time-efficient method of information retrieval for cancer patients.

In one aspect, the present disclosure provides a system for identifying at least one relevant subject for a cancer patient, the system comprising a server arrangement communicably coupled to a database arrangement comprising a plurality of records and a user device of the cancer patient, wherein the server arrangement is configured to:
create a profile for the cancer patient by receiving inputs from the user device;
map soft and hard attributes of the cancer patient with pre-existing profiles present in the database arrangement;
list the at least one relevant subject for the cancer patient; and
connect the cancer patient with the listed at least one subject for information sharing purposes.

Embodiments of the disclosure are advantageous in terms of providing an easy-to-use information retrieval system for cancer patients. Also, the system empowers patient(s) with information to navigate cancer journey. The system provides accurate information of cancer survivors based on the patient’s cancer profile and details entered in the system.

Optionally, the inputs received from the user device includes bibliographic information, cancer type, pre-existing conditions, treatment protocols and geographical location of the cancer patient.

Optionally, the soft attributes includes at least one of age, preferred location, cancer type, cancer sub-type, cancer stage, mutation, resection and severity.

Optionally, the hard attributes includes at least one of indication, gender, severity, Her-2 type, receptor type and cancer sub-type.

Optionally, the system further generates a relevancy score of each pre-existing profile for listing the at least one relevant subject.

In another aspect, the present disclosure provides a method for identifying at least one relevant subject for a cancer patient, the method comprising:
creating a profile for the cancer patient by receiving inputs from the user device;
mapping soft and hard attributes of the cancer patient with pre-existing profiles present in the database arrangement;
listing the at least one relevant subject for the cancer patient; and
connect the cancer patient with the listed at least one subject for information sharing purposes.

Optionally, the inputs received from the user device includes bibliographic information, cancer type, pre-existing conditions, treatment protocols and geographical location of the cancer patient.

Optionally, the soft attributes includes at least one of age, preferred location, cancer type, cancer sub-type, cancer stage, mutation, resection and severity.

Optionally, the hard attributes includes at least one of indication, gender, severity, Her-2 type, receptor type and cancer sub-type.

Optionally, the method further includes generating a relevancy score of each pre-existing profile for listing the at least one relevant subject.

Embodiments of the present disclosure substantially eliminate or at least partially address the aforementioned problems in the prior art, and provides a manageable and efficient method for identifying at least one relevant subject for a cancer patient.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary embodiments of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is an illustration of a network environment in which a system for identifying at least one relevant subject for a cancer patient is implemented, in accordance with an embodiment of the present disclosure; and

FIG. 2 is an illustration of steps of a method for identifying at least one relevant subject for a cancer patient, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item to which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

In one aspect, the present disclosure provides a system for identifying at least one relevant subject for a cancer patient, the system comprising a server arrangement communicably coupled to a database arrangement comprising a plurality of records and a user device of the cancer patient, wherein the server arrangement is configured to:
create a profile for the cancer patient by receiving inputs from the user device;
map soft and hard attributes of the cancer patient with pre-existing profiles present in the database arrangement;
list the at least one relevant subject for the cancer patient; and
connect the cancer patient with the listed at least one subject for information sharing purposes.

In another aspect, the present disclosure provides a method for identifying at least one relevant subject for a cancer patient, the method comprising:
creating a profile for the cancer patient by receiving inputs from the user device;
mapping soft and hard attributes of the cancer patient with pre-existing profiles present in the database arrangement;
listing the at least one relevant subject for the cancer patient; and
connect the cancer patient with the listed at least one subject for information sharing purposes.

The present disclosure provides a system and method of identifying at least one relevant subject for a cancer patient that is efficient in terms of time and processing power required for use thereof. The system and method of the present disclosure enable disambiguation of information relating to experience of a cancer survivor cancer including treatment protocols, therapies, clinical trials (existing and upcoming), and experts, thereby allowing an increased amount of information to be available for a cancer patient. Furthermore, the system significantly reduces entity recognition errors, ambiguous references. The system described herein de-duplicates repetitive information belonging to the same entity, thereby significantly reducing sizes of datasets and processing power required for processing thereof. Additionally, the method described herein does not require human intervention for functioning thereof. Furthermore, the method exhibits a very low computational (namely, processing) and time complexity.

Moreover, embodiments of the disclosure are advantageous in terms of providing an easy-to-use information retrieval system for cancer patients. Also, the system empowers patient(s) with information to navigate cancer journey. The system provides accurate information about experience of cancer survivor(s) to the patient diagnosed with cancer or undergoing the cancer treatment.

Moreover, information retrieval system is updated in real-time so that the most recently approved therapies and launched clinical trials are at patient’s fingertips.

The system comprises a server arrangement. Herein, the term “server arrangement” refers to a structure and/or module that include programmable and/or non-programmable components configured to store, process and/or share information. Optionally, the server arrangement includes any arrangement of physical or virtual computational entities capable of enhancing information to perform various computational tasks. Furthermore, it should be appreciated that the server may be both single hardware server and/or plurality of hardware servers operating in a parallel or distributed architecture. In an example, the server may include components such as memory, a processor, a network adapter and the like, to store, process and/or share information with other computing components, such as user device/user equipment. Optionally, the server is implemented as a computer program that provides various services (such as database service) to other devices, modules or apparatus.

The server arrangement is communicably coupled to a database arrangement. Herein, the term “database arrangement” refers to an organized body of digital information, regardless of the manner in which the data or the organized body thereof is represented. Optionally, the database may be hardware, software, firmware and/or any combination thereof. For example, the organized body of related data may be in the form of a table, a map, a grid, a packet, a datagram, a file, a document, a list or in any other form. The database includes any data storage software and systems, such as, for example, a relational database like IBM DB2 and Oracle 9.

The database arrangement stores a plurality of records. Herein, the term “record(s)” refers to electronic documents comprising information stored in a digital format. Notably, the information is recorded as a data type. Some examples of various data types are text data, tabular data, image data, and so forth. Thus, documents may be in any suitable file formats depending upon the data type in which the information is recorded. The records may include but not limited to bibliographic information, cancer type, pre-existing conditions, treatment protocols and geographical location of the cancer patient and cancer survivors. Further, the records include soft and hard attributes of the cancer patient and cancer survivors.

In an embodiment, the soft attributes includes at least one of age, preferred location, cancer type, cancer sub-type, cancer stage, mutation, resection and severity.

In another embodiment, the hard attributes includes at least one of indication, gender, severity, Her-2 type, receptor type and cancer sub-type.

The server arrangement is configured to generate an entity network by parsing the plurality of documents, wherein the entity network comprises a plurality of entities and their relationships, the plurality of entities comprising at least: document entities, name entities and topic entities. Herein, the term “entity” refers to an attribute of a document that provides characteristic information about the document. Examples of such characteristic information may include, but is not limited to, name of an author of the document, names of persons mentioned in the document, a unique identifier of the document, a topic to which the document belongs, content of the document, title of the document, publication organization from where the document originated, location of the publication organization. Therefore, attributes representing such characteristic information are extracted from the plurality of documents by parsing thereof and included in the entity network as entities. Specifically, parsing refers to analysing a document and determining syntactic roles of the content in the document using syntax analysis. Such syntactic analysis provides segregation of content in the document based on content type (such as cancer type, location and stage) and allow isolation of key information from the document. Furthermore, the server arrangement may parse metadata related to the document. Specifically, the metadata related to the document comprises tabulated information that is principal to the document.

The server arrangement and the database arrangement are communicably coupled to a user devices. Herein, the term “user device(s)” refers to a computing device and/or portable computing device. The computing device and/or portable computing device may include but not limited to a mobile device, a tablet and a personal computer.

In an embodiment, the information received for the user device includes bibliographic information, cancer type, pre-existing conditions and geographical location of the user. The bibliographic information may include but not limited to name, age, sex, height, weight and any other relevant information. The pre-existing conditions may include details related to existing medical conditions of the patient such as heart condition, blood pressure or any other information related to health condition of the patient(s).

The server arrangement is configured to generate an entity network by parsing the plurality of documents, wherein the entity network comprises a plurality of entities and their relationships, the plurality of entities comprising at least: document entities, name entities and topic entities. Herein, the term “entity” refers to an attribute of a document that provides characteristic information about the document. Specifically, parsing refers to analysing a document and determining syntactic roles of the content in the document using syntax analysis. Such syntactic analysis provides segregation of content in the document based on content type and allow isolation of key information from the document. Furthermore, the server arrangement may parse metadata related to the document. Specifically, the metadata related to the document comprises tabulated information that is principal to the document.

Optionally, extracting entities from the documents comprises cleaning and/or translating the documents. Specifically, cleaning the documents refers to removal of unnecessary comments, annotations, symbols, images and/or a combination thereof. Consequently, the server arrangement extracts only relevant information from the existing data sources. Moreover, translating the documents refers to conversion thereof to a machine-readable form. Beneficially, cleaning and/or translating the documents reduce processing complexity thereof. Additionally, cleaning and/or translating the documents also reduce processing time for identifying information relating to the entity. Optionally, a dedicated and adaptive subroutine may extract the information relating to the entities.

Optionally, the server arrangement is configured to determine, a relationship score of at least one relationship between a document entity and a name entity, based on classifiers of name entity that include at least one of: authored, mentioned. As mentioned previously, a name entity is representative of information relating to persons associated with the document.

Furthermore, the server arrangement is configured to identify relationships between the topic entities and at least one document entity. Specifically, relationships are identified between the document entities and the topic entities that are identified from those document entities. In an example, from a document represented by document entity ‘A’, identified topic entities are ‘artificial intelligence’ and ‘DNA sequencing’. Therefore, relationships between the topic entities, ‘artificial intelligence’ and ‘DNA sequencing’, and the document entity ‘A’ are established. Such identification of relationships is performed for every topic entity that is identified in each document from the plurality of documents.

The server arrangement is configured to determine a relevance score of at least one document entity based on relationships thereof with the name entities and the topic entities, and the importance score of each of the name entities using link analysis algorithm. Herein, the term “relevance score” as used herein the present disclosure relates to a measure of degree of relevance or significance of at least one document entity in the entity network.

In an embodiment, the system further generates a relevancy score of each pre-existing profile for listing the at least one relevant subject. Further, the relevancy score is obtained by using the following equation;

$$\begin{matrix}\left(0.55 * \left(1 - \text{score\_sub} \left[" \text{distance"}\right]\right)\right) + \left(0.25\right. \\ \text{Relevancy Score=} \left.* \text{score\_sub} \left[" \text{age"}\right]\right) + \left(0.15 * \text{score\_sub}\right. \\ \left.\left[" \text{stage"}\right]\right) \left(0.05 * \text{score\_sub} \left[" \text{country"}\right]\right)\end{matrix}$$

Distance: Maximum distance of the subject;
Age: Age of the subject;
Stage: Cancer stage of the subject; and
Country: Geographical location of the subject.

Table 1 provides details of the soft and the hard attributes of the cancer patients and the subject/cancer survivor(s) for various cancer types. Specifically, exact matching of hard attribute(s) of the cancer patients and the subject is required and scoring for the soft attributes is performed using the above-mentioned Eq. 1.

Table 1

| | | | |
|---|---|---|---|
| Cancer type | Attribute | Hard attribute - has to match | Soft attribute - scoring |
| General | Indication | × | |
| | Gender | × | |
| | Age | | × |
| | Preferred Area | | × |
| | Stage | | × |
| Breast | Severity | × | |
| | Mutations | | × |
| | Her2 | × | |
| | ER/PR | × | |
| | Lymph Node | × | |
| Prostate | Severity | × | |
| | Mutations | | × |
| Liver/Renal | Severity | × | |
| Colorectal | Severity | × | |
| | Mutations | | × |
| Lung | Histological type | × | |
| | Severity | × | |
| | Mutations | | × |
| Melanoma | Severity | × | |
| | Resection | | × |
| | Mutation | | × |
| Cholangio | Subtype(histological_type) | | × |
| | Severity | | × |
| | Mutation | | × |
| Pancreatic | Severity | | × |
| | Mutation | | × |

In an exemplary embodiment, various scores for soft attributes are as follows:

| | |
|---|---|
| Age | 20 |
| Preferred Area | 30 |
| Stage | 15 |
| country | 5 |
| Resection | 50 |
| Subtype | 60 |
| Mutation | 50 |

Based on the above mentioned attributes and using the Eq.1, the relevancy will be as follows:

| | | |
|---|---|---|
| Example | Subty | Relevancy |
| Patient A | Extrah | exact match |
| Patient B | Intrah | |
| Patient C | Klatskin | |
| Patient D | Klatskin | |

The present disclosure also relates to the method as described above. Various embodiments and variants disclosed above apply mutatis mutandis to the method.

Optionally, the inputs received from the user device includes bibliographic information, cancer type, pre-existing conditions, treatment protocols and geographical location of the cancer patient.

Optionally, the soft attributes includes at least one of age, preferred location, cancer type, cancer sub-type, cancer stage, mutation, resection and severity.

Optionally, the hard attributes includes at least one of indication, gender, severity, Her-2 type, receptor type and cancer sub-type.

Optionally, the method further includes generating a relevancy score of each pre-existing profile for listing the at least one relevant subject.

### DETAILED DESCRIPTION OF THE DRAWINGS

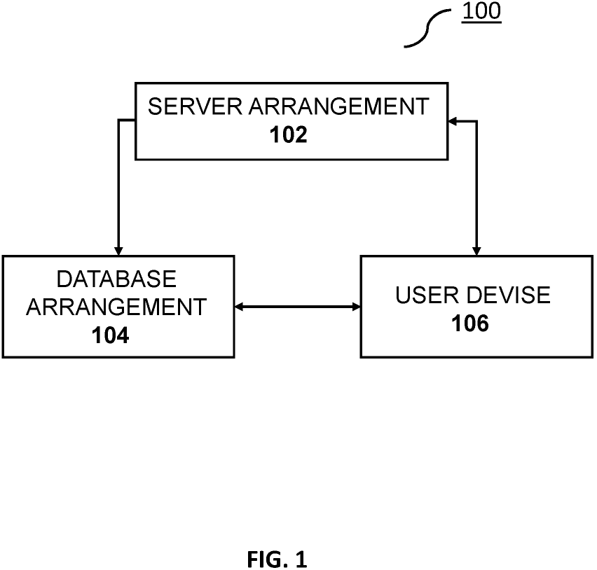

Referring to FIG. 1, there is shown a network environment 100 in which a system for identifying at least one relevant subject for a cancer patient is implemented, in accordance with an embodiment of the present disclosure. The system comprises a server arrangement 102 communicably coupled to a database arrangement 104 comprising a plurality of records and a user device 106, wherein the server arrangement 102 is configured to:
create a profile for the cancer patient by receiving inputs from the user device;
map soft and hard attributes of the cancer patient with pre-existing profiles present in the database arrangement;
list the at least one relevant subject for the cancer patient; and
connect the cancer patient with the listed at least one subject for information sharing purposes.

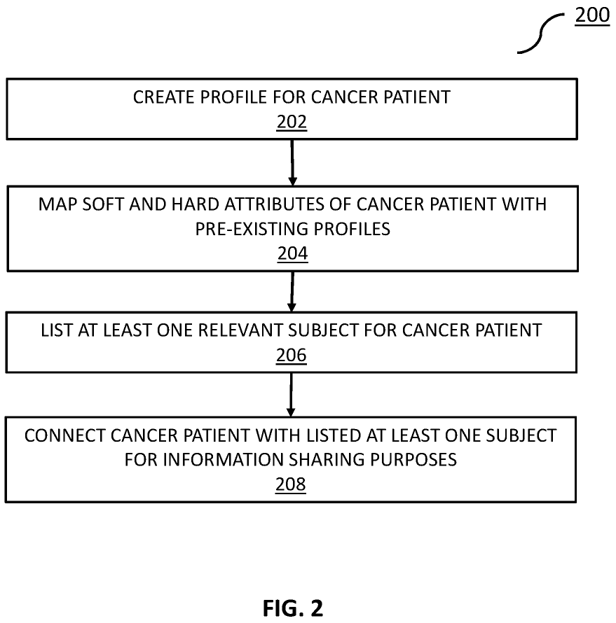

Referring to FIG. 2, illustrated are steps of a method 200 for identifying at least one relevant subject for a cancer patient, in accordance with an embodiment of the present disclosure. The method 200 is depicted as a collection of steps in a logical flow diagram, which represents a sequence of steps that can be implemented in hardware, software, or a combination thereof, for example as aforementioned. The method 200 is implemented using a system comprising a server arrangement communicably coupled to a database arrangement comprising a plurality of records and a user device. At a step 202, profile for a profile for the cancer patient is created by receiving inputs from the user device. At a step 204, soft and hard attributes of the cancer patient with pre-existing profiles present in the database arrangement are mapped. At a step 206, the at least one relevant subject for the cancer patient are listed. At a step 208, the cancer patient are connected with the listed at least one subject for information sharing purposes.

The steps 202 to 208 of method 200, are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural where appropriate.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

1. A system for identifying at least one relevant subject for a cancer patient, the system comprising a server arrangement communicably coupled to a database arrangement comprising a plurality of records and a user device of the cancer patient, wherein the server arrangement is configured to:
- create a profile for the cancer patient by receiving inputs from the user device;
map soft and hard attributes of the cancer patient with pre-existing profiles present in the database arrangement;
list the at least one relevant subject for the cancer patient; and
connect the cancer patient with the listed at least one subject for information sharing purposes.

2. A system of claim 1, wherein the inputs received from the user device includes bibliographic information, cancer type, pre-existing conditions, treatment protocols and geographical location of the cancer patient.

3. A system of claim 1, wherein the soft attributes includes at least one of age, preferred location, cancer type, cancer sub-type, cancer stage, mutation, resection and severity.

4. A system of claim 1, wherein the hard attributes includes at least one of indication, gender, severity, Her-2 type, receptor type and cancer sub-type.

5. A system of claim 1, wherein the system further generates a relevancy score of each pre-existing profile for listing the at least one relevant subject.

6. A method for identifying at least one relevant subject for a cancer patient, the method comprising:
creating a profile for the cancer patient by receiving inputs from the user device;
mapping soft and hard attributes of the cancer patient with pre-existing profiles present in the database arrangement;
listing the at least one relevant subject for the cancer patient; and
connect the cancer patient with the listed at least one subject for information sharing purposes.

7. A method of claim 6, wherein the inputs received from the user device includes bibliographic information, cancer type, pre-existing conditions, treatment protocols and geographical location of the cancer patient.

8. A method of claim 6, wherein the soft attributes includes at least one of age, preferred location, cancer type, cancer sub-type, cancer stage, mutation, resection and severity.

9. A method of claim 6, wherein the hard attributes includes at least one of indication, gender, severity, Her-2 type, receptor type and cancer sub-type.

10. A method of claim 6, wherein the method further includes generating a relevancy score of each pre-existing profile for listing the at least one relevant subject.
